(19) United States
(12) Patent Application Publication
(10) Pub. No.: US 2009/0306028 A1
(43) Pub. Date: Dec. 10, 2009

(54) COMPOSITIONS AND METHODS FOR TREATING INFLAMMATORY DISORDERS

(76) Inventor: Donald G. Payan, Hillsborough, CA (US)

Correspondence Address: Rigel Pharmaceuticals, Inc.;Bozicevic, Field & Francis LLP, 1900 University Ave, Suite 200, East Palo Alto, CA, 94303, US

(21) Appl. No.: 12/484,056

(22) Filed: Jun. 12, 2009

Related U.S. Application Data
(63) Continuation-in-part of application No. 11/555,222, filed on Oct. 31, 2006.
(60) Provisional application No. 60/732,452, filed on Oct. 31, 2005.

(51) Int. Cl. A61K 31/56; A61P 29/00

(52) U.S. Cl. 514/171

(57) ABSTRACT

The present disclosure relates to methods of treating inflammatory disorders by administering a Syk inhibitory 2,4-pyrimidinediamine compound and an anti-inflammatory agent.

### 1. CROSS REFERENCE TO RELATED APPLICATIONS

This application is a continuation in part of U.S. patent application Ser. No. 11/555,222, filed Oct. 31, 2006, which application claims benefit under 35 U.S.C. § 119(e) to U.S. application Ser. No. 60/732,452, filed Oct. 31, 2005, the contents of which are incorporated herein by reference.

### 2. TECHNICAL FIELD

The present disclosure relates to compositions and methods for treatment of inflammatory disorders.

### 3. BACKGROUND

Inflammation is a complex, stereotypical reaction of the body to damage inflicted on cells and vascularized tissues. The types of injury leading to an inflammatory reaction are varied, and include those induced by mechanical, physical, chemical, nutritive and biological insults. Injuries include those arising from traumatic force, radiation, heat, cold, toxins, irritants, oxygen deficiency, and infectious agents (e.g., viruses, microorganisms, protozoan and metazoan parasites). Inflammatory reaction may be also triggered by exogenous antigens, such as allergens, or genetic changes that cause destruction of cellular structures or defective activity of some enzymes and/or immune mediators.

The initial response of the body to tissue damage (acute inflammation) is vasodilation and increased capillary permeability due to alterations in the vascular endothelium. Changes in vascular permeability lead to increased blood flow (hyperemia) that causes redness (erythema) and the entry of fluid into the tissues (edema). As the inflammatory reaction progresses, there is recruitment of granulocytes, particularly neutrophils, into the tissues. The hallmark of this process is margination in which neutrophils attach to the endothelial cells within the blood vessels and then cross into the surrounding tissue (diapedesis), where under the influence of chemotactic factors, the cells become targeted to the locus of inflammation.

If the tissue damage is sufficiently severe, a chronic cellular response may follow, a phase characterized by the appearance of a mononuclear cell infiltrates composed of macrophages and lymphocytes. The macrophages are involved in microbial killing and removal of cellular and tissue debris. During this period, also known as resolution, normal tissue architecture may be restored while scarring may occur in other instances by in-filling with fibroblasts, collagen, and new endothelial cells. A granuloma is formed when macrophages and lymphocytes, together with epitheloid cells and gigant cells, accumulate around material that has not been eliminated. Angiogenesis may follow to revascularize new tissue and restore tissue function.

Inflammatory responses must be well ordered and controlled in order to limit the destructive cellular reaction and to localize the adverse consequences of the inflammatory response. However, persistence of inflammatory condition, such as from incomplete clearance of foreign material or repeated insults to a tissue, can lead to a chronic inflammatory state characterized by exaggerated tissue infiltration by mononuclear phagocytes and lymphocytes. Tissue destructive agents released by the macrophages, such as reactive oxygen species, can further damage the surrounding tissue and induce tissue remodeling, which can adversely affect tissue structure and function. The inflammatory process becomes self-perpetuating as tissue damage from the inflammatory response recruits additional macrophages and lymphocytes to the inflammatory site. In some instances, the balance of T-helper cell types (e.g., Th1 and Th2) becomes abnormal, which results in secretion of cytokines and inflammatory mediators that promote maintenance of the chronic inflammatory state by continued recruitment of macrophages and lymphocytes from the circulation, inducing their local proliferation and survival in the inflamed area.

Mounting evidence suggests that dysregulation of the inflammatory response is responsible for a variety of disease conditions, including atherosclerosis, rheumatoid arthritis, ulcerative colitis, Crohn's disease, and multiple sclerosis. Higher incidences of cancers in chronically inflamed tissues, such as cirrhotic liver in chronic hepatitis C virus infection or inflamed intestinal epithelia in Crohn's disease, suggests that prolonged inflammatory response may also induce genetic alterations responsible for abnormal cell proliferation. Thus, it is desirable to develop treatments to attenuate and/or localize the inflammatory reaction and ameliorate the adverse effects of a dysregulated inflammatory response.

### 4. SUMMARY

The present disclosure provides compositions and methods for treating inflammatory disorders. The compositions comprise a Syk inhibitory 2,4-pyrimidinediamine compound and an anti-inflammatory agent. The 2,4-pyrimidinediamine compounds inhibit or attenuate immune responses, such as an IgE mediated allergic response that are propagated via activation of IgE receptors or autoimmune responses mediated through IgG receptor. Without being limited by theory, the compounds appear to affect the immune response by inhibiting the activity of Syk kinase. In the present disclosure, these 2,4-pyrimidinediamine compounds are used in combination with anti-inflammatory agents, such as steroidal anti-inflammatory agents, non-steroidal anti-inflammatory agents, and anti-metabolites to treat inflammatory diseases.

In some embodiments, anti-inflammatory agents comprise steroidal anti-inflammatory agents, which include glucocorticosteroids and mineralocorticosteroids. These may be administered by any methods suitable for treating the inflammatory disorders, including, among others, oral, intravenous, intramuscular, dermal, or nasal routes.

In some embodiments, the anti-inflammatory agents comprise non-steroidal anti-inflammatory agents. These agents generally act by inhibiting the action of cyclooxygenase and lipoxygenase enzymes, or receptors for mediators generated by these enzymes. The non-steroidal anti-inflammatory compounds include non-selective COX inhibitors, selective COX inhibitors, as well as FLAP antagonists and 5-lipoxygenase antagonists.

In some embodiments, the anti-inflammatory agents can comprise anti-metabolites that affect proliferation of cells involved in the immune response. Suitable anti-metabolites include folate analogs, such as methotrexate; inosine monophosphate dehydrogenase (IMPDH) inhibitors, such as mycophenolate mofetil; and azathiopurine. Compounds of this group generally affect production of the substrates necessary for DNA replication, thereby inhibiting the proliferation of cells involved or activated in response to an inflammatory reaction.

In some embodiments, the 2,4-pyrimidinediamine compounds may be used with compatible combinations of anti-inflammatory agents, such as compatible combinations of steroidal and non-steroidal anti-inflammatory agents, steroidal and anti-metabolite agents, and non-steroidal and anti-metabolite agents. Identifying various combinations of exemplary compounds of each class will be apparent to the skilled artisan.

The 2,4-pyrimidinediamine compounds can be administered with the anti-inflammatory agent in the form of a composition, or administered adjunctively. When administered adjunctively, administration can be done sequentially or concurrently. Adjunctive administration may be by the same route or by different routes.

The compositions and methods may be used to treat a variety of inflammatory conditions. In some embodiments, the inflammatory condition treated is associated with an autoimmune disease, such a lupus erythematosus, multiple sclerosis, rheumatoid arthritis, and Crohn's disease. In other embodiments, the condition is an acute or chronic inflammatory condition, such as that associated with allergy, asthma, irritable bowel syndrome, ulcerative colitis, and psoriasis. In other embodiments, the condition is a malignancy, such as mastocytosis, fungoid mycosis (e.g., Sezary syndrome) and acute leukemia/lymphoma, which display inflammatory or allergic manifestations.

Further provided in the disclosure are kits comprising the 2,4-pyrimidinediamine compounds and the anti-inflammatory agent, combined together as a composition or as separate compositions for independent administration. The compounds may be provided in powders for reconstitution with a suitable solvent. In some embodiments, the kits can further comprise devices for administering a measured dose, examples of which include syringes, droppers or graduated cups. In some embodiments, the kits comprise a measured dosing device comprising the compound and the anti-inflammatory agent, such as metered dose device for administration by inhalation. The kit may also contain instructions in various mediums containing directions and guidance for dosing regimens and administration of the compounds.

### 5. DETAILED DESCRIPTION

### 5.1 Definitions

As used throughout the instant application, the following terms shall have the following meanings:

“Alkyl” by itself or as part of another substituent refers to a saturated or unsaturated branched, straight-chain or cyclic monovalent hydrocarbon radical having the stated number of carbon atoms (i.e., C1-C6 means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. In some embodiments, the alkyl group is (C1-C6) alkyl.

“Alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. In some embodiments, the alkanyl group is (C1-C6) alkanyl.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In some embodiments, the alkenyl group is (C2-C6) alkenyl.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In some embodiments, the alkynyl group is (C2-C6) alkynyl.

“Alkyldiyl” by itself or as part of another substituent refers to a saturated or unsaturated, branched, straight-chain or cyclic divalent hydrocarbon group having the stated number of carbon atoms (i.e., C1-C6 means from one to six carbon atoms) derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In some embodiments, the alkyldiyl group is (C1-C6) alkyldiyl. In some embodiments, the alkyldiyl groups are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In some embodiments, the alkyleno group is (C1-C6) or (C1-C3) alkyleno. In some embodiments, the alkyleno groups are straight-chain saturated alkano groups, e.g., methano, ethano, propano, butano, and the like.

“Heteroalkyl,” Heteroalkanyl,” Heteroalkenyl,” Heteroalkynyl,” Heteroalkyldiyl” and “Heteroalkyleno” by themselves or as part of another substituent refer to alkyl, alkanyl, alkenyl, alkynyl, alkyldiyl and alkyleno groups, respectively, in which one or more of the carbon atoms are each independently replaced with the same or different heteratoms or heteroatomic groups. Typical heteroatoms and/or heteroatomic groups which can replace the carbon atoms include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6) alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Acyclic Heteroatomic Bridge” refers to a divalent bridge in which the backbone atoms are exclusively heteroatoms and/or heteroatomic groups. Typical acyclic heteroatomic bridges include, but are not limited to, —O—, —S—, —S—O—, —NR′—, —PH—, —S(O)—, —S(O)₂—, —S(O) NR′—, —S(O)₂NR′—, and the like, including combinations thereof, where each R′ is independently hydrogen or (C1-C6) alkyl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like, as well as the various hydro isomers thereof.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In some embodiments, the aryl group is (C5-C15) aryl, with (C5-C10) being preferred. In some embodiments, the aryl groups are cyclopentadienyl, phenyl and naphthyl.

“Arylaryl” by itself or as part of another substituent refers to a monovalent hydrocarbon group derived by the removal of one hydrogen atom from a single carbon atom of a ring system in which two or more identical or non-identical parent aromatic ring systems are joined directly together by a single bond, where the number of such direct ring junctions is one less than the number of parent aromatic ring systems involved. Typical arylaryl groups include, but are not limited to, biphenyl, triphenyl, phenyl-naphthyl, binaphthyl, biphenyl-naphthyl, and the like. Where the number of carbon atoms in an arylaryl group are specified, the numbers refer to the carbon atoms comprising each parent aromatic ring. For example, (C5-C15) arylaryl is an arylaryl group in which each aromatic ring comprises from 5 to 15 carbons, e.g., biphenyl, triphenyl, binaphthyl, phenylnaphthyl, etc. In some embodiments, each parent aromatic ring system of an arylaryl group is independently a (C5-C15) aromatic. In some embodiments, more preferably a (C5-C10) aromatic. In some embodiments, the arylaryl groups are those in which all of the parent aromatic ring systems are identical, e.g., biphenyl, triphenyl, binaphthyl, trinaphthyl, etc.

“Biaryl” by itself or as part of another substituent refers to an arylaryl group having two identical parent aromatic systems joined directly together by a single bond. Typical biaryl groups include, but are not limited to, biphenyl, binaphthyl, bianthracyl, and the like. In some embodiments, the aromatic ring systems are (C5-C15) aromatic rings, while in other embodiments the aromatic ring systems are (C5-C10) aromatic rings. An exemplary biaryl group is biphenyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In some embodiments, the arylalkyl group is (C6-C21) arylalkyl, e.g., the alkanyl, alkenyl or alkynyl moiety of the arylalkyl group is (C1-C6) and the aryl moiety is (C5-C15). In some embodiments, the arylalkyl group is (C6-C13), e.g., the alkanyl, alkenyl or alkynyl moiety of the arylalkyl group is (C1-C3) and the aryl moiety is (C5-C10).

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), S(O)₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Specifically excluded from the definition of “parent heteroaromatic ring system” are benzene rings fused to cyclic polyalkylene glycols such as cyclic polyethylene glycols. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In some embodiments, the heteroaryl group is a 5-14 membered heteroaryl. In some embodiments, the heteroaryl group is a 5-10 membered heteroaryl.

“Heteroaryl-Heteroaryl” by itself or as part of another substituent refers to a monovalent heteroaromatic group derived by the removal of one hydrogen atom from a single atom of a ring system in which two or more identical or non-identical parent heteroaromatic ring systems are joined directly together by a single bond, where the number of such direct ring junctions is one less than the number of parent heteroaromatic ring systems involved. Typical heteroaryl-heteroaryl groups include, but are not limited to, bipyridyl, tripyridyl, pyridylpurinyl, bipurinyl, etc. Where the number of atoms are specified, the numbers refer to the number of atoms comprising each parent heteroaromatic ring systems. For example, 5-15 membered heteroaryl-heteroaryl is a heteroaryl-heteroaryl group in which each parent heteroaromatic ring system comprises from 5 to 15 atoms, e.g., bipyridyl, tripuridyl, etc. In some embodiments, each parent heteroaromatic ring system is independently a 5-15 membered heteroaromatic. In some embodiments, each parent heteroaromatic ring system is independently a 5-10 membered heteroaromatic. In some embodiments, the heteroaryl-heteroaryl groups are those in which all of the parent heteroaromatic ring systems are identical.

“Biheteroaryl” by itself or as part of another substituent refers to a heteroaryl-heteroaryl group having two identical parent heteroaromatic ring systems joined directly together by a single bond. Typical biheteroaryl groups include, but are not limited to, bipyridyl, bipurinyl, biquinolinyl, and the like. In some embodiments, the heteroaromatic ring systems are 5-15 membered heteroaromatic rings, more preferably 5-10 membered heteroaromatic rings.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6-21 membered heteroarylalkyl, e.g., the alkanyl, alkenyl or alkynyl moiety of the heteroarylalkyl is (C1-C6) alkyl and the heteroaryl moiety is a 5-15-membered heteroaryl. In particularly preferred embodiments, the heteroarylalkyl is a 6-13 membered heteroarylalkyl, e.g., the alkanyl, alkenyl or alkynyl moiety is (C1-C3) alkyl and the heteroaryl moiety is a 5-10 membered heteroaryl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

The above-defined groups may include prefixes and/or suffixes that are commonly used in the art to create additional well-recognized substituent groups. As examples, “alkyloxy” or “alkoxy” refers to a group of the formula —OR″, “alkylamine” refers to a group of the formula —NHR″ and “dialkylamine” refers to a group of the formula —NR″R″, where each R″ is independently an alkyl. As another example, “haloalkoxy” or “haloalkyloxy” refers to a group of the formula —OR′″, where R′″ is a haloalkyl.

“Substituted,” when used to modify a specified group or radical, means that one or more hydrogen atoms of the specified group or radical are each, independently of one another, replaced with the same or different substituent(s). Substituent groups useful for substituting for hydrogens on saturated carbon atoms in the specified group or radical include, but are not limited to —R⁶⁰, halo, —O⁻M⁺, ═O, —OR⁷⁰, —SR⁷⁰, —S⁻M⁺, ═S, —NR⁸⁰R⁸⁰, ═NR⁷⁰, ═N—OR⁷⁰, trihalomethyl, —CF₃, —CN, —OCN, —SCN, —NO, —NO₂, ═N₂, —N₃, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰C(O)O⁻M⁺, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of alkyl, cycloalkyl, heteroalkyl, cycloheteroalkyl, aryl, arylalkyl, heteroaryl and heteroarylalkyl; each R⁷⁰ is independently hydrogen or R⁶⁰; each R⁸⁰ is independently R⁷⁰ or alternatively, the two R⁸⁰'s, taken together with the nitrogen atom to which they are bonded, form a 5-, 6- or 7-membered cycloheteroalkyl which may optionally include from 1 to 4 of the same or different additional heteroatoms selected from the group consisting of O, N and S; and each M⁺ is a counter ion with a positive charge, for example, a positive charge independently selected from K⁺, Na⁺, ⁺N(R⁶⁰)₄, and Li⁺, or two of M⁺, combine to form a divalent counterion, for example a divalent counterion selected from Ca²⁺, Mg²⁺, and Ba²⁺. As specific examples, —NR⁸⁰R⁸⁰ is meant to include —NH₂, —NH-alkyl, N-pyrrolidinyl and N-morpholinyl.

Similarly, substituent groups useful for substituting for hydrogens on unsaturated carbon atoms in the specified group or radical include, but are not limited to, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰C(O)O⁻M⁺, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

Substituent groups, other than R^{P}, useful for substituting for hydrogens on nitrogen atoms in heteroalkyl and cycloheteroalkyl groups include, but are not limited to, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

Substituent groups from the above lists useful for substituting other groups or atoms specified as “substituted” will be apparent to those of skill in the art.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3 Ed., 1999, John Wiley & Sons, NY and Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated or alkylated such as benzyl and trityl ethers, as well as alkyl ethers, tetrahydropyranyl ethers, trialkylsilyl ethers (e.g., TMS or TIPPS groups) and allyl ethers.

“Prodrug” refers to a derivative of an active 2,4-pyrimidinediamine compound (drug) that requires a transformation under the conditions of use, such as within the body, to release the active 2,4-pyrimidinediamine drug. Prodrugs are frequently, but not necessarily, pharmacologically inactive until converted into the active drug. Prodrugs are typically obtained by masking a functional group in the 2,4-pyrimidinediamine drug believed to be in part required for activity with a progroup (defined below) to form a promoiety which undergoes a transformation, such as cleavage, under the specified conditions of use to release the functional group, and hence the active 2,4-pyrimidinediamine drug. The cleavage of the promoiety may proceed spontaneously, such as by way of a hydrolysis reaction, or it may be catalyzed or induced by another agent, such as by an enzyme, by light, by acid or base, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

A wide variety of progroups, as well as the resultant promoieties, suitable for masking functional groups in the active 2,4-pyrimidinediamines compounds to yield prodrugs are well-known in the art. For example, a hydroxyl functional group may be masked as a sulfonate, ester or carbonate promoiety, which may be hydrolyzed in vivo to provide the hydroxyl group. An amino functional group may be masked as an amide, carbamate, imine, urea, phosphenyl, phosphoryl or sulfenyl promoiety, which may be hydrolyzed in vivo to provide the amino group. A carboxyl group may be masked as an ester (including silyl esters and thioesters), amide or hydrazide promoiety, which may be hydrolyzed in vivo to provide the carboxyl group. Nitrogen protecting groups and nitrogen pro-drugs of the invention may include lower alkyl groups as well as amides, carbamates, etc. Other specific examples of suitable progroups and their respective promoieties will be apparent to those of skill in the art.

“Progroup” refers to a type of protecting group that, when used to mask a functional group within an active 2,4-pyrimidinediamine drug to form a promoiety, converts the drug into a prodrug. Progroups are typically attached to the functional group of the drug via bonds that are cleavable under specified conditions of use. Thus, a progroup is that portion of a promoiety that cleaves to release the functional group under the specified conditions of use. As a specific example, an amide promoiety of the formula —NH—C(O)CH₃ comprises the progroup —C(O)CH₃.

“Fc Receptor” refers to a member of the family of cell surface molecules that binds the Fc portion (containing the specific constant region) of an immunoglobulin. Each Fc receptor binds immunoglobulins of a specific type. For example the Fcα receptor (“FcαR”) binds IgA, the FcεR binds IgE and the FcγR binds IgG.

The FcαR family includes the polymeric Ig receptor involved in epithelial transport of IgA/IgM, the mycloid specific receptor RcαRI (also called CD89), the Fcα/μR and at least two alternative IgA receptors (for a recent review see Monteiro and van de Winkel, 2003, Annu. Rev. Immunol. 21:177-204. The FcαRI is expressed on neutrophils, eosinophils, moncytes/macrophages, dendritic cells and kupfer cells. The FcαRI includes one alpha chain and the FcR gamma homodimer that bears an activation motif (ITAM) in the cytoplasmic domain and phosphorylates Syk kinase.

The FcεR family includes two types, designated FcεRI and FcεRII (also known as CD23). FcεRI is a high affinity receptor (binds IgE with an affinity of about 10¹⁰M⁻¹) found on mast, basophil and eosinophil cells that anchors monomeric IgE to the cell surface. The FcεRI possesses one alpha chain, one beta chain and the gamma chain homodimer discussed above. The FcεRII is a low affinity receptor expressed on mononuclear phagocytes, B lymphocytes, eosinophils and platelets. The FcεRII comprises a single polypeptide chain and does not include the gamma chain homodimer.

The FcγRII family includes three types, designated FcγRIII (also known as CD64), FcγRII (also known as CD32) and FcγRIII (also known as CD16), and FcγRIV. FcγRI is a high affinity receptor (binds IgG1 with an affinity of 10⁸M⁻¹) found on mast, basophil, mononuclear, neutrophil, eosinophil, deudritic and phagocyte cells that anchors nomomeric IgG to the cell surface. The FcγRI includes one alpha chain and the gamma chain dimer shared by FcαRI and FcεRI.

The FcγRII is a low affinity receptor expressed on neutrophils, monocytes, eosinophils, platelets and B lymphocytes. The FcγRII includes one alpha chain, and does not include the gamma chain homodimer discussed above.

The FcγRIII is a low affinity (binds IgG1 with an affinity of 5×10⁵M⁻¹) expressed on NK, eosinophil, macrophage, neutrophil and mast cells. It comprises one alpha chain and the gamma homodimer shared by FcαRI, FcεRI and FcγRI.

The FcγRIV binds to IgG2a and IgG2b with intermediate affinity, and is expressed by myeloid lineage cells. FcγRIV maps on the 75 kb genomic interval between FcγRII and FcγRIII (see Ravetch et. al., 2005, Immunity 23:41-51).

Skilled artisans will recognize that the subunit structure and binding properties of these various Fc receptors, cell types expressing them, are not completely characterized. The above discussion merely reflects the current state-of-the-art regarding these receptors (see, e.g., Immunobiology: The Immune System in Health & Disease, 5th Edition, Janeway et al., Eds, 2001, ISBN 0-8153-3642-x, FIG. 9.30 at pp. 371), and is not intended to be limiting with respect to the myriad receptor signaling cascades that can be regulated with the compounds described herein.

“Fc Receptor-Mediated Degranulation” or “Fc Receptor-Induced Degranulation” refers to degranulation that proceeds via an Fc receptor signal transduction cascade initiated by crosslinking of an Fc receptor.

“IgE-Induced Degranulation” or “FcεRI-Mediated Degranulation” refers to degranulation that proceeds via the IgE receptor signal transduction cascade initiated by crosslinking of FcεR1-bound IgE. The crosslinking may be induced by an IgE-specific allergen or other multivalent binding agent, such as an anti-IgE antibody. In mast and/or basophil cells, the FcεRI signaling cascade leading to degranulation may be broken into two stages: upstream and downstream. The upstream stage includes all of the processes that occur prior to calcium ion mobilization. The downstream stage includes calcium ion mobilization and all processes downstream thereof. Compounds that inhibit FcεRI-mediated degranulation may act at any point along the FcεRI-mediated signal transduction cascade. Compounds that selectively inhibit upstream FcεRI-mediated degranulation act to inhibit that portion of the FcεRI signaling cascade upstream of the point at which calcium ion mobilization is induced. In cell-based assays, compounds that selectively inhibit upstream FcεRI-mediated degranulation inhibit degranulation of cells such as mast or basophil cells that are activated or stimulated with an IgE-specific allergen or binding agent (such as an anti-IgE antibody) but do not appreciably inhibit degranulation of cells that are activated or stimulated with degranulating agents that bypass the FcεRI signaling pathway, such as, for example the calcium ionophores ionomycin and A23187.

“IgG-Induced Degranulation” or “FcγRI-Mediated Degranulation” refers to degranulation that proceeds via the FcγRI signal transduction cascade initiated by crosslinking of FcγRI-bound IgG. The crosslinking may be induced by an IgG-specific allergen or another multivalent binding agent, such as an anti-IgG or fragment antibody. Like the FcεRI signaling cascade, in mast and basophil cells the FcγRI signaling cascade also leads to degranulation which may be broken into the same two stages: upstream and downstream. Similar to FcεRI-mediated degranulation, compounds that selectively inhibit upstream FcγRI-mediated degranulation act upstream of the point at which calcium ion mobilization is induced. In cell-based assays, compounds that selectively inhibit upstream FcγRI-mediated degranulation inhibit degranulation of cells such as mast or basophil cells that are activated or stimulated with an IgG-specific allergen or binding agent (such as an anti-IgG antibody or fragment) but do not appreciably inhibit degranulation of cells that are activated or stimulated with degranulating agents that bypass the FcγRI signaling pathway, such as, for example the calcium ionophores ionomycin and A23187.

“Ionophore-Induced Degranulation” or “Ionophore-Mediated Degranulation” refers to degranulation of a cell, such as a mast or basophil cell, that occurs upon exposure to a calcium ionophore such as, for example, ionomycin or A23187.

“Syk Kinsase” refers to the well-known 72 kDa non-receptor (cytoplasmic) spleen protein tyrosine kinase expressed in B-cells and other hematopoetic cells. Syk kinase includes two consensus Src-homology 2 (SH2) domains in tandem that bind to phosphorylated immunoreceptor tyrosine-based activation motifs (“ITAMs”), a “linker” domain and a catalytic domain (for a review of the structure and function of Syk kinase see Sada et al., 2001, J. Biochem. (Tokyo) 130:177-186); see also Turner et al., 2000, Immunology Today 21:148-154). Syk kinase has been extensively studied as an effector of B-cell receptor (BCR) signaling (Turner et al., 2000, supra). Syk kinase is also critical for tyrosine phosphorylation of multiple proteins which regulate important pathways leading from immunoreceptors, such as Ca²⁺ mobilization and mitogen-activated protein kinase (MAPK) cascades and degranulation. Syk kinase also plays a critical role in integrin signaling in neutrophils (see, e.g., Mocsai et al. 2002, Immunity 16:547-558).

As used herein, Syk kinase includes kinases from any species of animal, including but not limited to, homosapiens, simian, bovine, porcine, rodent, etc., recognized as belonging to the Syk family. Specifically included are isoforms, splice variants, allelic variants, mutants, both naturally occurring and man-made. The amino acid sequences of such Syk kinases are well known and available from GENBANK. Specific examples of mRNAs encoding different isoforms of human Syk kinase can be found at GENBANK accession no. gi|21361552|ref|NM_{—}003177.2|, gi|496899|emb|Z29630.1|HSSYKPTK[496899] and gi|15030258|gb|BC011399.1|BC011399[15030258], which are incorporated herein by reference.

Skilled artisans will appreciate that tyrosine kinases belonging to other families may have active sites or binding pockets that are similar in three-dimensional structure to that of Syk. As a consequence of this structural similarity, such kinases, referred to herein as “Syk mimics,” are expected to catalyze phosphorylation of substrates phosphorylated by Syk. Thus, it will be appreciated that such Syk mimics, signal transduction cascades in which such Syk mimics play a role and biological responses effected by such Syk mimics and Syk mimic-dependent signaling cascades may be regulated, and in particular inhibited, with the 2,4-pyrimidinediamine compounds described herein.

“Syk-Dependent Signaling Cascade” refers to a signal transduction cascade in which Syk kinase plays a role. Non-limiting examples of such Syk-dependent signaling cascades include the FcαRI, FcεRI, FcγRI, FcγRIII, BCR and integrin signaling cascades.

“Autoimmune Disease” refers to those diseases which are commonly associated with the nonanaphylactic hypersensitivity reactions (Type II, Type III and/or Type IV hypersensitivity reactions) that generally result as a consequence of the subject's own humoral and/or cell-mediated immune response to one or more immunogenic substances of endogenous and/or exogenous origin. Such autoimmune diseases are distinguished from diseases associated with the anaphylactic (Type I or IgE-mediated) hypersensitivity reactions.

“Inflammatory Response” or “Inflammatory Reaction” refers to a physiologic reaction initiated by a diverse array of stimuli, including infectious agents, antigen-antibody reactions, physical injury, and autoimmune activity. The clinical condition of inflammation is characterized by the presence of erythema, edema, hyperalgesia, and pain. Recognized phases of an inflammatory reaction include (1) an acute transient phase characterized by vasodilation and enhanced capillary permeability, (2) a delayed, subacute phase characterized by infiltration of leukocytes and phagocytic cells, and (3) a chronic proliferative phase characterized by tissue degeneration and tissue remodeling (e.g., fibrosis).

Presence of certain cellular mediators can also characterize the inflammatory response, including mediators such as proinflammatory cytokines (e.g., TNF-α, IL-1, IL-2, IL-8, IL-17, IFN-γ, etc.), lipid mediators, (e.g., prostaglandins PGE₂ and PGI₂; leukotriene B4, etc.); and small molecules mediators (e.g., NO). These mediators affect various phases of the inflammatory response, such as vascular permeability, and mobilization, recruitment, proliferation, and activation of macrophages, granulocytes, and T-lymphocytes. Without being bound by theory, it is believed that some inflammatory disorders arise, in part, from an imbalance of T-helper cell types, Th1 and Th2, which produce different subsets of cytokines to activate humoral or cell-mediated immune responses. For instance, abnormal humoral response in asthma is correlated elevated activity of Th2 helper cells while abnormal cell mediated immune response in Crohn's disease is correlated with elevated activity of Th1 helper cells.

“Inflammatory Disorder” or “Inflammatory Disease” refers to dysregulated inflammatory reaction that causes an exaggerated response by macrophages, granulocytes, and/or T-lymphocytes leading to abnormal tissue damage and cell death. The heightened inflammatory response may ultimately lead to tissue reorganization and compromised tissue function. Exemplary inflammatory diseases include inflammatory bowel disease, psoriasis, and atherosclerosis. In some instances, the inflammatory disorder is a byproduct of other primary dysfunction in immune system function, such as autoimmune disease and allergic response.

### 5.2 2,4-Pyrimidinediamine Compounds and Anti-Inflammatory Agents

The present disclosure provides compositions and methods for treating inflammatory conditions and diseases, using a combination of compounds that affect different cellular processes involved in immune function. The combinations comprise a Syk inhibitory 2,4-pyrimidinediamine compound characterized by its ability to inhibit or attenuate the Syk dependent signaling cascade though inhibition of Syk, a protein kinase present in hematopoietic cells and differentiated cells of the hematopoietic lineage, such as B-cells and T-cells (see, e.g., Couture et al., 1994, Proc Natl Acad Sci USA 91(12):5301-5; Law et al., 1994, J Biol. Chem. 269(16):12310-9; Thome et al., 1995, J Exp Med. 181(6):1997-2006; Latour et al., 1997, Mol Cell Biol. 17(8):4434-41; and Mustelin T and Tasken K., 2003, Biochem J. 371(Pt 1):15-27). The Syk inhibitory compound is administered with an anti-inflammatory agent that modulates the immune response, generally by a different therapeutic mechanism than the Syk inhibitor. Suitable anti-inflammatory agents include, among others, steroidal anti-inflammatory agents, non-steroidal anti-inflammatory agents, β-adrenergic receptor agonists, and anti-metabolites affecting development and proliferation of immune system cells. By using a combination of therapeutic agents working through different mechanisms, more effective treatments may be obtained as compared to use of a single therapeutic compound.

5.2.1 Syk Inhibitory 2,4-Pyrimidinediamine Compounds

The Syk inhibitory 2,4-pyrimidinediamine compounds for use in treating inflammatory disorders are described in U.S. published patent application Nos. 2004/0029902, 2005/0038243, 2005/0209224, 2005/0209230, 2005/0234049; PCT published applications WO 2005/016893, WO 2005/01399, and WO 2004/014382; U.S. application Ser. No. 10/631,029, filed Jul. 29, 2003; and U.S. Provisional Application Ser. No. 60/630,808, filed Nov. 24, 2004; all disclosures of which are incorporated herein by reference in their entirety. The 2,4-pyrimidinediamine compounds are potent inhibitors of degranulation of immune cells, such as mast, basophil, neutrophil and/or eosinophil cells. While not intending to be bound by any theory, the 2,4-pyrimidinediamine compounds appears to exert their degranulation inhibitory effect, at least in part, by blocking or inhibiting the signal transduction cascade(s) initiated by crosslinking of the high affinity Fc receptors for IgE (“FcRI”) and/or IgG (“FcγRI”). It is believed that this inhibition of cellular degranulation and/or the release of other chemical mediators occur primarily by inhibiting Syk kinase. Other types of receptors acting through Syk include, among others, T cell receptors, Epstein Barr virus protein 2A, and TNF receptors TNFR1 and TNFR-2 (see, e.g., Muljo, S. A. and Schlissel M. S., 2000, Immunol Rev. 175:80-93; Takada, Y. and Aggarwal, B. B., 2004, J. Immunol. 173(2): 1066-77). Because Syk kinase appears to be intimately involved with a myriad of other immune functions, such as T and B cell development, inhibitiors of Syk kinase find use in modulating other aspects of the immune system function, such as the inflammatory reaction.

Generally, the 2,4-pyrimidinediamine compounds that are capable of inhibiting Syk kinase comprise a 2,4-pyrimidinediamine “core” having the following structure and numbering convention:

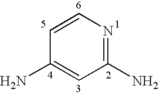

The compounds are substituted at the C2 nitrogen (N2) to form a secondary amine and are optionally further substituted at one or more of the positions at the C4 nitrogen (N4), the C5 position and/or the C6 position. When substituted at N4, the substituent forms a secondary amine. The substituent at N2, as well as the optional substituents at the other positions, may range broadly in character and physicochemical properties. For example, the substituent(s) may be a branched, straight-chained or cyclic alkyl, a branched, straight-chained or cyclic heteroalkyl, a mono- or polycyclic aryl a mono- or polycyclic heteroaryl or combinations of these groups. These substituent groups may be further substituted as described in the references cited above.

The N2 and/or N4 substituents may be attached directly to their respective nitrogen atoms, or they may be spaced away from their respective nitrogen atoms via linkers, which may be the same or different. The nature of the linkers can vary widely, and can include virtually any combination of atoms or groups useful for spacing one molecular moiety from another. For example, the linker may be an acyclic hydrocarbon bridge (e.g., a saturated or unsaturated alkyleno such as methano, ethano, etheno, propano, prop[1]eno, butano, but[1]eno, but[2]eno, buta[1,3]dieno, and the like), a monocyclic or polycyclic hydrocarbon bridge (e.g., [1,2]benzeno, [2,3]naphthaleno, and the like), a simple acyclic heteroatomic or heteroalkyldiyl bridge (e.g., —O—, —S—, —S—O—, —NH—, —PH—, —C(O)—, —C(O)NH—, —S(O)—, —S(O)₂—, —S(O)NH—, —S(O)₂NH—, —O—CH₂—, —CH₂—O—CH₂—, —O—CH═CH—CH₂—, and the like), a monocyclic or polycyclic heteroaryl bridge (e.g., [3,4]furano, pyridino, thiopheno, piperidino, piperazino, pyrazidino, pyrrolidino, and the like) or combinations of such bridges.

The substituents at the N2, N4, C5 and/or C6 positions, as well as the optional linkers, may be further substituted with one or more of the same or different substituent groups. The nature of these substituent groups may vary broadly. Non-limiting examples of suitable substituent groups include branched, straight-chain or cyclic alkyls, mono- or polycyclic aryls, branched, straight-chain or cyclic heteroalkyls, mono- or polycyclic heteroaryls, halos, branched, straight-chain or cyclic haloalkyls, hydroxyls, oxos, thioxos, branched, straight-chain or cyclic alkoxys, branched, straight-chain or cyclic haloalkoxys, trifluoromethoxys, mono- or polycyclic aryloxys, mono- or polycyclic heteroaryloxys, ethers, alcohols, sulfides, thioethers, sulfanyls (thiols), imines, azos, azides, amines (primary, secondary and tertiary), nitriles (any isomer), cyanates (any isomer), thiocyanates (any isomer), nitrosos, nitros, diazos, sulfoxides, sulfonyls, sulfonic acids, sulfamides, sulfonamides, sulfamic esters, aldehydes, ketones, carboxylic acids, esters, amides, amidines, formadines, amino acids, acetylenes, carbamates, lactones, lactams, glucosides, gluconurides, sulfones, ketals, acetals, thioketals, oximes, oxamic acids, oxamic esters, etc., and combinations of these groups. Substituent groups bearing reactive functionalities may be protected or unprotected, as is well-known in the art.

In some embodiments, the 2,4-pyrimidinediamine comprise compounds according to structural formula (I):

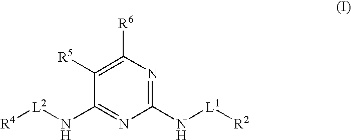

including salts, hydrates, solvates and N-oxides thereof, wherein:

L¹ and L² are each, independently of one another, selected from the group consisting of a direct bond and a linker;

R² is selected from the group consisting of (C1-C6) alkyl optionally substituted with one or more of the same or different R⁸ groups, (C3-C8) cycloalkyl optionally substituted with one or more of the same or different R⁸ groups, cyclohexyl optionally substituted with one or more of the same or different R⁸ groups, 3-8 membered cycloheteroalkyl optionally substituted with one or more of the same or different R⁸ groups, (C5-C15) aryl optionally substituted with one or more of the same or different R⁸ groups, phenyl optionally substituted with one or more of the same or different R⁸ groups and 5-15 membered heteroaryl optionally substituted with one or more of the same or different R⁸ groups;

R⁴ is selected from the group consisting of hydrogen, (C1-C6) alkyl optionally substituted with one or more of the same or different R⁸ groups, (C3-C8) cycloalkyl optionally substituted with one or more of the same or different R⁸ groups, cyclohexyl optionally substituted with one or more of the same or different R⁸ groups, 3-8 membered cycloheteroalkyl optionally substituted with one or more of the same or different R⁸ groups, (C5-C15) aryl optionally substituted with one or more of the same or different R⁸ groups, phenyl optionally substituted with one or more of the same or different R⁸ groups and 5-15 membered heteroaryl optionally substituted with one or more of the same or different R⁸ groups;

R⁵ is selected from the group consisting of R⁶, (C1-C6) alkyl optionally substituted with one or more of the same or different R⁸ groups, (C1-C4) alkanyl optionally substituted with one or more of the same or different R⁸ groups, (C2-C4) alkenyl optionally substituted with one or more of the same or different R⁸ groups and (C2-C4) alkynyl optionally substituted with one or more of the same or different R⁸ groups;

each R⁶ is independently selected from the group consisting of hydrogen, an electronegative group, —OR^{d}, —SR^{d}, (C1-C3) haloalkyloxy, (C1-C3) perhaloalkyloxy, —NR^{c}R^{c}, halogen, (C1-C3) haloalkyl, (C1-C3) perhaloalkyl, —CF₃, —CH₂CF₃, —CF₂CF₃, —CN, —NC, —OCN, —SCN, —NO, —NO₂, —N₃, —S(O)₂R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)NR^{c}R^{c}, —OS(O)₂NR^{c}R^{c}, —C(O)R^{d}, —C(O)OR^{d}, —C(O)NR^{c}R^{c}, —C(NH)NR^{c}R^{c}, —OC(O)R^{d}, —SC(O)R^{d}, —OC(O)OR^{d}, —SC(O)OR^{d}, —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙNR^{c}R^{c} and —[NHC(NH)]ₙNR^{c}R^{c}, (C5-C10) aryl optionally substituted with one or more of the same or different R⁸ groups, phenyl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups and 6-16 membered heteroarylalkyl optionally substituted with one or more of the same or different R⁸ groups;

R⁸ is selected from the group consisting of R^{a}, R^{b}, R^{a} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —NH[(CH₂)ₘR^{b}], —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};

each R^{a} is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b} is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};

each R^{c} is independently R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which is optionally substituted with one or more of the same or different R^{a} or suitable R^{b} groups;

each R^{d} is independently R^{a};

each m is independently an integer from 1 to 3; and

each n is independently an integer from 0 to 3.

In some embodiments, the 2,4-pyrimidinediamine compounds of structural formula (I) above comprise compounds in which L¹ and L² are each a direct bond;

R² is selected from the group consisting of phenyl mono substituted at the 3- or 5-position with an R⁸ group, phenyl di- or tri-substituted with one or more of the same or different R⁸ groups and 5-15 membered heteroaryl optionally substituted with one or more of the same or different R⁸ groups;

R⁴ is selected from the group consisting of phenyl substituted with one or more of the same or different R⁸ groups and 5-15 membered heteroaryl optionally substituted with one or more of the same or different R⁸ groups;

R⁵ is selected from the group consisting of —CN, —NC, —NO₂, fluoro, (C1-C3) haloalkyl, (C1-C3) perhaloalkyl, (C1-C3) fluoroalkyl, (C1-C3) perfluoroalkyl, —CF₃, (C1-C3) haloalkoxy, (C1-C3) perhaloalkoxy, (C1-C3) fluoroalkoxy, (C1-C3) perfluoroalkoxy, —OCF₃, —C(O)R^{a}, —C(O)OR^{a}, —C(O)CF₃ and —C(O)OCF₃;

R⁶ is hydrogen;

R⁸ is selected from the group consisting of R^{e}, R^{b}, R^{e} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘ—R^{b}, —S—(CH₂)ₘ—R^{b} —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)—C(O)NH—(CH₂)ₙ—R^{b}, —S—(CH₂)ₘ—C(O)NH—(CH₂)ₘ—R^{b}, —O—(CHR^{a})—C(O)NH—(CHR^{a})—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —NH[(CH₂)ₘR^{b}], —N[(CH₂)ₘR^{b}]₂, —NH—C(O)NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};

each R^{a} is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b} is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d} —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};

each R^{c} is independently a protecting group or R^{a}, or, alternatively, two R^{c} are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different R^{a} groups;

each R^{d} is independently a protecting group or R^{a};

each R^{e} is independently selected from the group consisting of (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each m is independently an integer from 1 to 3; and

each n is independently an integer from 0 to 3, with the provisos that:
(1) when R² is a substituted phenyl, then R⁵ is other than cyano; and
(2) when R² and R⁴ are each independently a substituted or unsubstituted pyrrole or indole, then the R² and R⁴ are attached to the remainder of the molecule via a ring carbon atom.

In some embodiments of the 2,4-pyrimidinediamine compounds above, R² is selected from the group consisting of phenyl, 5-10 membered heteroaryl, benzodioxanyl, 1,4-benzodioxan-(5 or 6)-yl, benzodioxolyl, 1,3-benzodioxol-(4 or 5)-yl, benzoxazinyl, 1,4-benzoxazin-(5,6,7 or 8)-yl, benzoxazolyl, 1,3-benzoxazol-(4,5,6 or 7)-yl, benzopyranyl, benzopyran-(5,6,7 or 8)-yl, benzotriazolyl, benzotriazol-(4,5,6 or 7)-yl, 1,4-benzoxazinyl-2-one, 1,4-benzoxazin-(5,6,7 or 8)-yl-2-one, 2H-1,4-benzoxazinyl-3(4H)-one, 2H-1,4-benzoxazin-(5,6,7 or 8)-yl-3(4H)-one, 2H-1,3-benzoxazinyl-2,4(3H)-dione, 2H-1,3-benzoxazin-(5,6,7 or 8)-yl-2,4(3H)-dione, benzoxazolyl-2-one, benzoxazol-(4,5,6 or 7)-yl-2-one, dihydrocoumarinyl, dihydrocoumarin-(5,6,7 or 8)-yl, 1,2-benzopyronyl, 1,2-benzopyron-(5,6,7 or 8)-yl, benzofuranyl, benzofuran-(4,5,6 or 7)-yl, benzo[b]furanyl, benzo[b]furan-(4,5,6 or 7)-yl, indolyl, indol-(4,5,6 or 7)-yl, pyrrolyl and pyrrol-(1 or 2)-yl, each of which may be optionally substituted with one or more of the same or different R⁸ groups, where R⁸ is as defined above.

Specific embodiments of Syk kinase inhibitory 2,4-pyrimidinediamine compounds are described in Appendixes A, B, C and D of U.S. provisional application Ser. No. 60/690,351, filed Jun. 13, 2005. Compounds useful in the methods described herein also include 2,4-pyrimidinediamine compounds described in U.S. application Ser. No. 10/355,543 (U.S. application publication No. 2004/0029902), including the exemplary 2,4-pyrimidinediamine compounds of Examples 7.3.1 to 7.3.1098, compounds of Example 7.3.1099, and compounds of Examples 7.3.1100 to 7.3.1165; U.S. application Ser. No. 10/631,029, filed Jul. 29, 2003, and corresponding PCT publication WO 2004/014382, including each of specific compounds disclosed as Examples 7.3.1 to 7.3.1165 and Examples 7.4.1 to 7.4.445; U.S. application Ser. Nos. 10/903,263 and 10/903,870, concurrently filed Jul. 30, 2004 (U.S. application publication No. 2005/0234049 and 2005/0209224, respectively), including each of specific compounds described in Table I (i.e., compound numbers 200 to 1358); and U.S. Application Ser. No. 60/630,808, filed Nov. 24, 2004. All publications, patent applications, and specific compounds are incorporated herein by reference in their entirety. The 2,4-pyrimidinediamine compounds useful for the purposes herein further include salts, hydrates, solvates, N-oxides, and prodrugs of the Syk inhibitory compounds.

As noted above, in some embodiments, the Syk inhibitory compounds can comprise prodrugs of the biologically active 2,4-pyrimidinediamine compounds. In some embodiments, the Syk inhibitory compounds comprise prodrugs described in U.S. application Ser. No. 10/355,543 (U.S. application publication No. 2004/0029902); U.S. application Ser. No. 10/631,029, filed Jul. 29, 2003, and corresponding PCT publication WO2004/014382; and U.S. application Ser. No. 11/337,049 and corresponding international application PCT/US2006/001945, filed concurrently on Jan. 19, 2006, entitled “Prodrugs of 2,4-pyrimidinediamine compounds and their uses,” all of which are incorporated herein by reference.

In some embodiments, the prodrugs include such active 2,4-pyrimidinediamine compounds in which one or more of the available primary or secondary amine groups is masked with a progroup R^{P} that metabolizes in vivo to yield the active 2,4-pyrimidinediamine drug. The nature of the prodrug can vary, and will depend upon, among other factors, the desired water solubility of the prodrug, its intended mode of administration, and or its intended mechanism or site of metabolism to the active 2,4-pyrimidinediamine compound.

In some embodiments, the prodrug forms of the active 2,4-pyrimidinediamine compounds include pyrimidinediamines in which the N4-substituent of the 2,4-pyrimidine moiety is a substituted or unsubstituted nitrogen-containing heteroaryl ring of the formula (II):

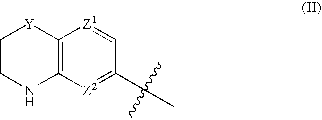

wherein Z¹ and Z² are each, independently of one another, selected from CH and N and Y is selected from CH₂, NH, O, S, S(O) and S(O)₂. Such prodrugs can include progroups R^{P} at: one or both of the non-aromatic ring nitrogens of the heteroaryl ring, the N2-nitrogen of the 2,4-pyrimidinediamine moiety, the N4-nitrogen atom of the 2,4-pyrimidinediamine moiety and/or any available nitrogen atoms in the substituent attached to the N2 nitrogen atom of the 2,4-pyrimidinediamine moiety.

In some embodiments, the prodrugs of 2,4-pyrimidinediamines comprise compounds according to the following structural formula (III):

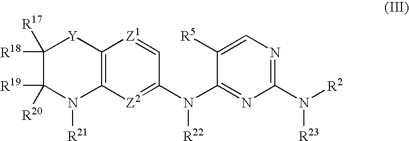

including salts, solvates, hydrates and N-oxides thereof, wherein:

Y is selected from CH₂, NR²⁴, O, S, S(O) and S(O)₂;

Z¹ and Z² are each, independently of one another, selected from CH and N;

R² is selected from lower alkyl optionally substituted with one or more of the same or different R⁸ groups, lower cycloalkyl optionally substituted with one or more of the same or different R⁸ groups, cyclohexyl optionally substituted with one or more of the same or different R⁸ groups, 3-8 membered cycloheteroalkyl optionally substituted with one or more of the same or different R⁸ groups, (C6-C14) aryl optionally substituted with one or more of the same or different R⁸ groups, phenyl optionally substituted with one or more of the same or different R⁸ groups and 5-15 membered heteroaryl optionally substituted with one or more of the same or different R⁸ groups;

R⁵ is selected from halo, fluoro, cyano, nitro, trihalomethyl and trifluoromethyl;

R⁸ is selected from R^{a}, R^{b}, R^{a} substituted with one or more, for example, from one to four, of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘ—R^{b} —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —NH[(CH₂)ₘR^{b}], —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};

R¹⁷ is selected from hydrogen, halogen, fluoro, lower alkyl and methyl or, alternatively, R¹⁷ may be taken together with R¹⁸ to form an oxo (═O) group or, together with the carbon atom to which they are attached, a spirocycle containing from 3 to 7 carbon atoms;

R¹⁸ is selected from hydrogen, halogen, fluoro, lower alkyl and methyl or, alternatively, R¹⁸ may be taken together with R¹⁷ to form an oxo (═O) group or, together with the carbon atom to which they are attached, a spirocycle containing from 3 to 7 carbon atoms;

R¹⁹ is selected from hydrogen, lower alkyl, and methyl or, alternatively, R¹⁹ may be taken together with R²⁰ to form an oxo (═O) group or, together with the carbon atom to which they are attached, a spirocycle containing from 3 to 7 carbon atoms;

R²⁰ is selected from hydrogen, lower alkyl and methyl or, alternatively, R²⁰ may be taken together with R¹⁹ to form an oxo (═O) group or, together with the carbon atom to which they are attached, a spirocycle containing from 3 to 7 carbon atoms;

each R^{a} is, independently of the others, selected from hydrogen, lower alkyl, lower cycloalkyl, cyclohexyl, (C4-C11) cycloalkylalkyl, (C6-C10) aryl, phenyl, (C7-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b} is a suitable group independently selected from ═O, —OR^{a}, (C1-C3) haloalkyloxy, ═S, —SR^{a}, ═NR^{a}, ═NOR^{a}, —NR^{c}R^{c}, halogen, —CF₃, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)R^{a} —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{a}, —OC(O)OR^{a}, —OC(O)NR^{c}R^{c}, —OC(NH)NR^{c}R^{c}, —OC(NR^{a})NR^{c}R^{c}, —[NHC(O)]ₙR^{a}, —[NR^{a}C(O)]ₙR^{a}, —[NHC(O)]ₙOR^{a}, —[NR^{a}C(O)]ₙOR^{a}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};

each R^{c} is, independently of the others, selected from a protecting group and R^{a}, or, alternatively, the two R^{c} bonded to the same nitrogen atom are taken together with that nitrogen atom to form a 5 to 8-membered cycloheteroalkyl or heteroaryl which may optionally include one or more of the same or different additional heteroatoms and which may optionally be substituted with one or more, for example, from one to four, of the same or different R^{a} groups;

R²¹, R²² and R²³ are each, independently of one another, selected from hydrogen and a progroup R^{P};

R²⁴ is selected from hydrogen, lower alkyl and progroup R^{P};

each m is, independently of the others, an integer from 1 to 3; and

each n is, independently of the others, an integer from 0 to 3, with the proviso that at least one of R²¹, R²², R²³ and R²⁴ is a progroup.

In the prodrugs useful for the purposes herein, and in particular in the prodrugs of structural formula (III), R²¹, R²² and R²³ each represents either hydrogen or a progroup R^{p}. Also, R²⁴ represents hydrogen, a lower alkyl or a progroup R^{P}. Thus, the prodrugs can include a single R^{P} progroup, two R^{P} progroups, three R^{P} progroups, or even more R^{P} progroups, depending, in part, on the identity of Y and whether the R² substituent includes any R^{P} progroups. In some embodiments, it is preferred that the prodrugs described herein, and in particular the prodrugs of structural formula (III), include only one R^{P} group. Without intending to be bound by any theory of operation, it is possible that the different R^{P} groups in prodrugs including more than one R^{P} progroup may metabolize at different rates. Prodrugs including a single R^{P} progroup would avoid such differential metabolic kinetics. A specific embodiment of prodrugs according to structural formula above that include a single progroup R^{P} are compounds according to the following structural formula (IV):

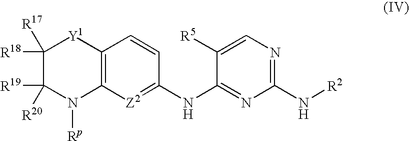

wherein Y¹ is selected from CH₂, NR²⁴, O, S, S(O) and S(O)₂; and Z², R², R⁵, R¹⁷, R¹⁸, R¹⁹, R²⁰, R²⁴ and R^{P} are as previously defined, with the proviso that R² does not include any R^{P} groups.

In some embodiments, the progroup R^{P} includes at least one of an ester, a thioester, an ether, a thioether, a silyl ether, a thiosilyl ether, a carbonate, a thiourea, an amide, a thioamide, a carbamate and a urea linkage. In some embodiments, the progroup R^{P} comprises a phosphate group, such as a phosphate ester.

In embodiments where the R^{P} comprises a phosphate group, R^{P} has the formula —(CR^{d}R^{d})_{y}—O—P(O)(OH)₂, or a salt thereof, where y is an integer ranging from 1 to 3; each R^{d} is, independently of the others, selected from hydrogen, optionally substituted lower alkyl, optionally substituted (C6-C14) aryl and optionally substituted (C7-C20) arylalkyl; where the optional substituents are, independently of one another, selected from hydroxyl, lower alkoxy, (C6-C14) aryloxy, lower alkoxyalkyl, methoxymethyl, methoxyethyl, ethoxymethyl, ethoxyethyl and halogen, or, alternatively, two R^{d} bonded to the same carbon atom are taken together with the cabon atom to which they are bonded to form a cycloalkyl group containing from 3 to 8 carbon atoms. In some embodiments, the progroup R^{P} is selected from —CH₂—O—P(O)(OH)₂ and —CH₂CH₂—O—P(O)(OH₂), and salts thereof.

In some embodiments, the progroup R^{P} is selected from —(CR^{d}R^{d})y-O—P(O)(OR^{e})(OH), —(CR^{d}R^{d})y-O—P(O)(OR^{e})(OR^{e}),

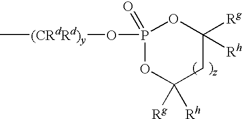

and salts thereof, wherein each R^{e} is, independently of the others, selected from substituted or unsubstituted lower alkyl, substituted or unsubstituted (C6-C14) aryl (e.g., phenyl, naphthyl, 4-loweralkoxyphenyl, 4-methoxyphenyl), substituted or unsubstituted (C7-C20) arylalkyl (e.g., benzyl, 1-phenylethan-1-yl, 2-phenylethan-1-yl), —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f} and —Si(R^{d})₃, wherein each R^{f} is, independently of the others, selected from hydrogen, unsubstituted or substituted lower alkyl, substituted or unsubstituted (C6-C14) aryl, and substituted or unsubstituted (C7-C20) arylalkyl;

each R^{g} is, independently of the others, selected from hydrogen and lower alkyl; each R^{h} is, independently of the others, selected from hydrogen, substituted or unsubstituted lower alkyl, substituted or unsubstituted lower cycloheteroalkyl, substituted or unsubstituted (C6-C14) aryl, substituted or unsubstituted (C7-C20) arylalkyl and substituted or unsubstituted 5-14 membered heteroaryl; z is an integer ranging from 0 to 2; and R^{d} and y are as defined above.

Exemplary prodrug compounds useful in the methods herein include specific compounds disclosed in Example 7.4 of U.S. application Ser. No. 10/355,543 (U.S. application publication No. 2004/0029902), each of specific compounds disclosed in Examples 7.4.1 to 7.4.445 of U.S. application Ser. No. 10/631,029, filed Jul. 29, 2003, and corresponding PCT publication WO2004/014382; and Examples 7.1, 7.2, 7.3, and 7.4 of U.S. application Ser. No. 11/337,049 and corresponding international application PCT/US2006/001945 discussed above.

Depending upon the nature of the various substituents, the 2,4-pyrimidinediamine compounds and prodrugs may be in the form of salts. Such salts include salts suitable for pharmaceutical uses (“pharmaceutically-acceptable salts”), and salts suitable for veterinary uses, etc. Such salts may be derived from acids or bases, as is well-known in the art.

In various embodiments, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, cycloalkylsulfonic acids (e.g., camphorsulfonic acid), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion or an aluminum ion), an ammonium ion or coordinates with an organic base (e.g., ethanolamine, diethanolamine, triethanolamine, N-methylglucamine, morpholine, piperidine, dimethylamine, diethylamine, etc.).

The 2,4-pyrimidinediamine compounds may be administered individually or as compatible combinations along with the anti-inflammatory agent. Different combinations of the 2,4-pyrimidinediamine compounds may be used to adjust bioavailability, duration of effect, and efficacy for the particular inflammatory condition. Identifying appropriate combinations for the purposes herein are within the skill of those in the art.

5.2.2 Steroidal Anti-inflammatory Agents

For treating inflammatory disorders, the Syk inhibitory 2,4-pyrimidinediamine compounds are administered in combination with an anti-inflammatory agent. In some embodiments, the anti-inflammatory agent comprises a steroidal anti-inflammatory agent. As used herein, “steroidal anti-inflammatory agent” or “anti-inflammatory steroid” comprises a compound or composition based on a structure with a steroid nucleus and having anti-inflammatory activity, either alone or in combination with other agents. With the exception of vitamin D compounds, steroid compounds are derived from a steroid nucleus based on a saturated tetracyclic hydrocarbon, 1,2-cyclopentanoperhydrophenanthrene, also referred to as sterane or gonane. Steroidal compounds include both naturally occurring and synthetically produced steroidal compounds. Different groups of steroid compounds include, among others, adrenocorticosteroids, estrogens/progestins, and androgens.

In some embodiments, the steroidal anti-inflammatory agents are adrenocorticosteroids, which refer to steroidal compounds that are released from the adrenal cortex. These steroid compounds include the groups of glucocorticosteroids and mineralocorticosteoids. As used herein, adrenocorticosteroids also include various synthetic analogs that display the biological properties displayed by the naturally occurring steroids. Certain structural features may enhance anti-inflammatory activities of steroids, such as all trans steroid skeleton, presence of Δ⁴-3-keto, 11β-OH, 17β-OH, and substitutions at 9α-, 6α-, 16α-positions, with F>Cl>Br>I.

In some embodiments, the anti-inflammatory steroidal agent is a glucocorticosteroid (synonymously “glucocorticoid”). At the physiological level, glucocorticosteroids affect glucose metabolism by stimulating gluconeogenesis, mobilization of amino acids from extrahepatic tissues, and stimulation of fat breakdown in adipose tissue. The anti-inflammatory effects of glucocorticoids are believed to arise by their effect on synthesis of the genes involved in production of inflammatory mediators and by limiting proliferation of T cells, which in some cases arise from induction of T cell apoptosis. Various anti-inflammatory glucocorticoids can be used. These include, by way of example and not limitation, natural and synthetic steroidal compounds such as 21-acetoxypregnenolone, alclometasone, algestone, amcinonide, beclomethasone, budesonide, chloroprednisone, ciclesonide, clobetasol, clobetasone, clocortolone, cloprednol, corticosterone, cortisone, contrivazol, deflazacort, desonide, desoximetasone, dexamethansone, diflorasone, diflucortolone, difluprednate, enoxolone, fluazacort, flurandrenolone acetonide, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinode, halobetasol propionate, halometasone, halopredone acetate, hydrocortamate, hydrocortisone, hydrocortisone 17-butyrate, hydrocortisone 17-valerate, loteprednol etabonate, maziprednone, medrysone, mepredinsone, methylprednisolone, mometasone furoate, paramethasone, prednicarbate, prednisolone, prednisolone 21-dimethylaminoacetate, prenisolone sodium phosphate, prednisone, prednival, prednylidene, rimexolone, tixocortol, triamcinolone, triamcinolone acetonide, triamcinolone benetonide, and triamcinolone hexacetonide. Other glucocorticosteroids will be apparent to the skilled artisan.

In some embodiments, the anti-inflammatory steroid is a mineralocorticosteroid (synonymously “mineralocorticoid”). At the physiological level, mineralocorticoids affect water and electrolyte balance, particularly through their effect on ion transport in renal tubules, resulting in retention of sodium and loss of potassium. Various mineralocorticoids include, among others, aldosterone, deoxycorticosterone, deoxycorticosterone acetate, and fludrocortisone. It is to be understood, however, that the characterization of a steroid as a glucocorticosteroid or mineralocorticosteroid are used for descriptive purposes and is not meant to be exclusionary. Glucocorticoids display some mineralocorticosteroid activity while some mineralocorticoids display some glucocorticoid activity. For the purposes herein, a mineralocorticoid with anti-inflammatory properties may be used. Generally, mineralocorticosteroids with some glucocorticosteroid activity appears to have anti-inflammatory effects. An exemplary anti-inflammatory mineralocorticoid is fludrocortisone.

In some embodiments, the anti-inflammatory steroidal agents have varying biologic effect half-life, and can be divided into short acting, intermediate acting, or long acting steroidal compounds (see, e.g., Liapi C. and Chrousos G. P., “Glucocorticoids,” in Therapeutic Principles in Practice. 2nd Ed., 1992, (Jaffe, S. J. and Aranda J. V., eds.), pgs. 466-475, Philadelphia, Pa., W B Saunders; “Adrenal Cortical Steroids,” in Drug Facts and Comparisons, 5th Ed., 1997, pg 122-128, St. Louis, Mo., Facts and Comparisons, Inc.); and Goodman & Gilman's The Pharmacological Basis of Therapeutics, 2001, pg 1657, 10^{th} Ed. (Hardman et al, eds.), McGraw Hill).

Generally, a short-acting anti-inflammatory steroidal compound displays a biologic half-life of about 8 to about 12 hrs. Exemplary short-acting steroidal compounds include, by way of example and not limitation, cortisol and cortisone.

In some embodiments, the anti-inflammatory steroid is an intermediate acting compound, which generally displays a biologic half-life of about 12 to about 36 hrs. Exemplary intermediate-acting steroidal compounds include, by way of example and not limitation, prednisone, prednisolone, triamcinolone, and methylprednisolone.

In some embodiments, the anti-inflammatory steroid is a long-acting steroidal compound, which generally displays a biologic half-life of about 36 to about 72 hrs. Exemplary long-acting steroidal compounds include, by way of example and not limitation, dexamethasone, betamethasone, and budesonide.

In other embodiments, the anti-inflammatory steroid is an antedrug, which refers to an active synthetic derivative that is designed to undergo biotransformation to the readily excretable inactive form upon entry in the systemic circulation. Antedrugs are generally applied topically and act locally to minimize systemic side effects and increase the therapeutic indices. An exemplary steroid of this type is steroid-21-oate esters as described in Khan et al., 2005, Curr Med Chem. 12(19):2227-39. This compound quickly hydrolyzes to the corresponding inactive steroid acids, thereby exerting minimal systemic side effects. Other steroidal antedrugs are described in Khan, supra; Kwon et al., 1995, J Med Chem 38:1048-1051; Park et al., 2003, Steroids 68:315-319; Druzgala et al., 1991, J Steroid Biochem Mol Biol 38:149-154; Ueno et al., 1991, J Med Chem 34:2468-2473; Chanoine et al., 1991, Drug Metab Dispos 19:546-553; Moodley et al., 1991, J Lipid Mediat 3:51-70; Milioni et al., 1991, Eur J Med Chem 26:947-951; and Biggadike et al., 2000, J Med Chem 43:19-21). All disclosures incorporated herein by reference.

In other embodiments, the anti-inflammatory steroid is a nitro-steroidal compound. As used herein a “nitro-steroidal” compound is steroid having NO-releasing activity (the nitrosterols), and include NO-releasing forms of prednisolone, flunisolide and hydrocortisone. These steroids are thought to provide more potent anti-inflammatory activity than their parent molecules when tested in animal models of acute and chronic inflammation. Steroidal anti-inflammatory compounds of this group are described in Doggrell, S. A., 2005, Expert Opinion on Investigational Drugs 14(7):823-828), incorporated herein by reference.

In some embodiments, the steroidal anti-inflammatory agent can be an inhaled steroidal agent, which is useful for nasal administration and/or absorption through the lungs. These forms are effective agents for treating asthma and reaction to inhaled allergens. Various forms of steroidal anti-inflammatory compounds formulated as inhalants include, among others, beclomethasone, bedesonide, dexamethasone, flunisolide, triamcinolone acetonide, and antedrugs noted above.

In some embodiments, the steroidal anti-inflammatory agent is an estrogen or a synthetic estrogen analog. Mounting evidence suggests that estrogen and estrogen analogs attenuate the onset of the inflammatory reaction in several animal models of inflammation-associated pathological conditions (see, e.g., Ghisletti et al., 2005, Mol Cell Biol. 25(8):2957-68). In addition, estrogen deficiency may render postmenopausal women vulnerable to degenerative conditions such as arthritis, atherosclerosis, and Alzheimer's disease, all disorders in which inflammation is implicated in the etiology of the disease state. Various estrogen and estrogen analogs that may be used include, by way of example and not limitation, estrogen, 17 β-estradiol, estrogen conjugates, medroxyprogesterone, 2-methoxyestradiol (estrogen metabolite), diethystilbesterol, reveratrol, phytoestrogens (e.g., genestein), and tamoxifen.

In other embodiments, the steroidal anti-inflammatory compound comprises vitamin D and analogs thereof. Various anti-inflammatory agents of this group include, by way of example and not limitation, 7-dehydrocholesterol, cholecaciferol, ergosterol, 1,25-dihydroxyvitamin D3, and 22-ene-25-oxa-vitamin D. Other vitamin D analogs are described in U.S. Pat. Nos. 6,924,400; 6,858,595; 6,689,922; and 6,573,256.

5.2.3 Non-Steroidal Anti-inflammatory Agents

In some embodiments, the anti-inflammatory agent is a non-steroidal anti-inflammatory agent (NSAID). This class of agents comprises a heterogeneous group of compounds with varying structures but which act through common therapeutic targets. While not being bound by any theories on the mechanism of action, NSAIDs exert their biological effects by affecting the synthesis of prostaglandins by cyclooxygenase (COX), also referred to as prostaglandins endoperoxidase synthase, which transforms arachidonic acid to intermediates prostaglandin G2 and prostaglandin H2. Two forms of the enzyme have been identified. COX-1 is found in most cell types and is expressed constitutively. COX-2 is inducible by the action of various cytokines and inflammatory mediators, but is also expressed constitutively in some tissues. Because COX-1 expression in the stomach is constitutive as opposed to COX-2, inhibitors of COX-2 is indicated as having less gastric toxicity as compared to inhibitors of COX-1. In addition, COX-2 is believed to be the isoform that is the major pathway for synthesis of proinflammatory prostaglandins. Prostaglandins that are ultimately produced from the intermediates generated by cyclooxygenase activity mediate a variety of inflammatory responses. For instance, prostaglandin E2 (PGE₂) and prostacyclin (PG1₂) causes vasodilation and consequently increase in blood flow and erythema.

NSAIDs are classified based on their chemical structures and biological activities. In some embodiments, the NSAIDs useful with the 2,4-pyrimidinediamine compounds are non-selective COX-2 inhibitors, which inhibit the activity of both COX-1 and COX-2 isoforms. The prototypical non-selective COX inhibitor is salicylic acid and derivatives thereof. Exemplary compounds of this class include, by way of example and not limitation, acetylsalicylic acid, sodium salicylate, choline magnesium trisalicylate, salsalate, diflunisal, sulfasalazine, olsalazine, and mesalamine.

In some embodiments, the non-selective COX inhibitors are indole and indene acetic acids. Exemplary compounds of this class include, among others, indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac.

In other embodiments, the non-selective COX inhibitors comprise heteroaryl acetic acids. Exemplary compounds of this class include, among others, tolmetin, diclofenac, and ketorolac.

In still other embodiments, the non-selective COX inhibitors comprise arylpropionic acids or propionic acid derivatives (profens). Exemplary compounds of this class include among others, alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen.

In still other embodiments, the non-selective COX inhibitors comprise anthranilic acids (fenamates). Exemplary compounds of this class include, among others, flufenamic acid, meclofenamic acid, mefenamic acid, niflumic acid and tolfenamic acid.

In still other embodiments, the non-selective COX inhibitors comprise enolic acids (e.g., oxicams). Exemplary compounds of this class include, among others, piroxicam and meloxicam, isoxicam, and sudoxicam and tenoxican.

In still other embodiments, the non-selective COX inhibitors comprise phenylpyrazolones. Exemplary compounds of this class include, among others, phenylbutazone, apazone, bezpiperylon, feprazone, mofebutazone, oxyphenbutazone.

In still other embodiments, the non-selective COX inhibitors comprise biphenylcarboxylic acid derivatives. Exemplary compounds of this class include, among others, diflunisal and flufenisal.

In some embodiments, the NSAIDs are selective COX-2 inhibitors. As used herein, a selective COX-2 inhibitor preferably inhibits the activity of COX-2 isozyme as compared to the inhibition of the COX-1 isozyme. A selective COX-2 inhibitor can have a selectivity (i.e., inhibition of COX-2/COX-1) of about 10, of about 20 of about 50, of about 100, of about 200, of about 500, and of about 1000 or more. Selectivity is based on assay typically used to measure COX activity.

In some embodiments, the selective COX-2 inhibitor comprises diaryl-substituted furanones. An exemplary compound of this class includes, among others, refocoxib, available under the tradename Vioxx®.

In other embodiments, the selective COX-2 inhibitor comprises diaryl-substituted pyrazoles. An exemplary compound of this class includes, among others, celecoxib, available under the tradename Celebrex®.

In still other embodiments, the selective COX-2 inhibitor comprises indole acetic acids. An exemplary compound of this class includes, among others, etodolac, available under the tradename Lodine®.

In still other embodiments, the selective COX-2 inhibitor comprises sulfonanilides. An exemplary compound of this class includes, among others, nimesulide.

The NSAIDs, including pharmaceutically acceptable salts thereof, may be used alone or as compatible combinations of NSAIDs.

5.2.4 Lipoxygenase and 5-Lipoxygenase activating protein (FLAP) antagonists

In some embodiments, the non-steroidal anti-inflammatory agent that may be used with the 2,4-pyrimidinediamine compounds is a lipoxygenase or a 5-lipoxygenase activating protein (FLAP) antagonist. Lipoxygenases are a family of non-heme iron containing dioxygenases catalyzing the oxygenation of arachidonic acid to generate leukotrienes (LT) and hydroxyeicosatetraenoic acid (HETE). LT production is catalyzed by 5-lipoxygenase (5-LP) in presence of FLAP to generate 5-hydroperoxyeicosatetraenoic acid. Through additional transformations of 5-hydroperoxyeicosatetraenoic acid, leukotrienes LTC₄, LTD₄ and LTE₄ are formed. These have biological effects as slow-acting mediators of anaphylaxis.

In some embodiments, various antagonists of lipoxygenase may be used to ameliorate the inflammatory response mediated by the leukotrienes. Classes of lipoxygenase inhibitors include, among others, N-hydroxyurea derivatives, redox inhibitors, and non-redox inhibitors. Exemplary N-hydroxyurea derived inhibitors include, by way of example and not limitation, 1-(1-benzothiophen-2-ylethyl)-1-hydroxy-urea (leutrol), 1-[4-[5-(4-fluorophenoxy)-2-furyl]but-3-yn-2-yl]-1-hydroxy-urea; 1-[(2R)-4-[5-[(4-fluorophenyl)methyl]thiophen-2-yl]but-3-yn-2-yl]-1-hydroxy-urea (atreleuton); 3-(1-benzothiophen-2-ylethyl)-1-hydroxy-urea (see, e.g., Steele et al., 1999, Cancer Epidemiol Biomarkers Prev. 8(5):467-83, the disclosure of which incorporated herein by reference). An exemplary redox inhibitor includes, by way of example and not limitation, 2-(12-hydroxydodeca-5,10-diynyl)-3,5,6-trimethyl-cyclohexa-2,5-diene-1,4-dione (docebenone). An exemplary non-redox inhibitor includes, by way of example and not limitation, 6-[[3-fluoro-5-(4-methoxyoxan-4-yl)phenoxy]methyl]-1-methyl-quinolin-2-one (i.e., ZD2138).

In other embodiments, a FLAP antagonist may be used as the anti-inflammatory agent. FLAP antagonists include, among others, indole derivatives and qunoline derivatives. Exemplary indole derivatives with FLAP inhibitory activity include, by way of example and not limitation, 3-[3-butylsulfanyl-1-[(4-chlorophenyl)methyl]-5-propan-2-yl-indol-2-yl]-2,2-dimethyl-propanoic acid (i.e., MK-866) and 3-[1-[(4-chlorophenyl)methyl]-5-(quinolin-2-ylmethoxy)-3-tert-butylsulfanyl-indol-2-yl]-2,2-dimethyl-propanoic acid (i.e., MK0591 or quiflapon). Exemplary quinoline derivatives include, by way of example and not limitation, (2R)-2-cyclopentyl-2-[4-(quinolin-2-ylmethoxy)phenyl]acetic acid (i.e., BAY-X1005 and veliflapon) (Steele et al., supra).

5.2.5 Anti-Histamines

In some embodiments, the 2,4-pyrimidinediamine compounds are used in combination with anti-histamines, which are generally H1-receptor antagonists. H1 antagonists typically inhibit histamine action on smooth muscle, vasodilation, and granule release from mast cells and basophils. Various classes of H1-receptor agonists include compounds based on, among others, tricyclic dibenzoxepins, ethanolamines, ethylenediamines, alkylamines, piperazine, phenothiazines, piperidines, and phthalazinones.

Exemplary H1 receptor antagonists include, among others, doxepin, cabinoxamine, clemastine, diphenylhydramine, dimenhydrinate, pyrilamine, tripelennamine, chlorpheniramine, bromopheniramine, hydroxyzine, cyclizine, meclizine, promethazine, cyproheptadine, phenindamine, acrivastine, citirizine, azelastine, levocabastine, loratadine, fexofenadine, and various salts, hydrates, N-oxides, and prodrugs thereof.

Other embodiments of various anti-histamine compounds will be apparent to the skilled artisan.

5.2.6 β-Agonists

In other embodiments, the 2,4-pyrimidinediamine compounds are used in combination with β-adrenergic receptor agonists (synonymously “β-agonists” or “β-adrenergic agonists”), which includes non-selective β-adrenergic agonists as well as β₂-selective adrenergic agonists. There are generally two types of β-agonists. Short-acting β-agonists display an onset of action that begins within minutes of administration and lasts for approximately 2 to 6 hrs. The long-acting β-adrenergic agonists displays activity that lasts for 12 hrs or more, and are generally highly specific to the β₂-adrenergic receptor.

Exemplary short acting β-adrenergic agonists include, by way of example and not limitation, albuterol (salbutamol), isotharine, fenoterol, levalbuterol, metaproterenol (orciprenaline), procaterol, terbutaline, and pirbuterol. In various embodiments, these agents may be provided in inhaled as well as oral dosage forms. Exemplary long-acting β-adrenergic agonists include, by way of example and not limitation, salmeterol xinafoate, formoterol, and bitolterol. Although selective β₂-adrenergic agonists are typically used, non-selective β-adrenergic agonists may be used for systemic applications. Exemplary non-selective β-agonists include, by way of example and not limitation, isoproterenol and dobutamine.

5.2.7 Anti-Metabolite Anti-Inflammatory Agents

In some embodiments, the anti-inflammatory agent is an anti-metabolite that attenuates or inhibits the activation and/or proliferation of cells involved in inflammation. Anti-metabolites may have cytostatic or cytotoxic effects and thus generally display immunosuppressive characteristics.

Various anti-inflammatory anti-metabolites may be used in combination with the 2,4-pyrimidinediamine compounds. In some embodiments, the anti-proliferative agent comprises methotrexate, a folate analogue that competitively binds and inhibits dihydrofolate reductase (DHFR), and thus inhibits the synthesis of thymidine and other compounds requiring methylation through single carbon transfer reactions.

In other embodiments, the anti-proliferative anti-metabolite comprises an inhibitor of inosine monophosphate dehydrogenase (IMPDH), the enzyme acting in the salvage pathway for the synthesis of guanosine monophosphate (GMP) from inosine. GMP is an essential nucleoside for purine synthesis during cell division, and although most cell types are capable of synthesizing GMP de novo, T and B-lymphocytes almost exclusively use the salvage pathway of purine synthesis, and are thus sensitive to the inhibitory action of these compounds. IMPDH inhibitors useful as anti-inflammatory agents include, among others, mycophenolic acid, mycophenolate mofetil, ribavirin, taizofurin, selenazofurin, benazamide adenine dinucleotide, and benzamide riboside (see, e.g. Pankiewicz and Goldstein, Inosine Monophosphate Dehydrogenase: A Major Therapeutic Target, 2003, American Chemical Society; U.S. Pat. No. 6,867,299; U.S. Pat. No. 6,713,623; U.S. Pat. No. 5,932,600; and U.S. Pat. No. 5,493,030). Other IMPDH inhibitors will be apparent to the skilled artisan.

Other exemplary anti-metabolites include azathioprine, 6-mercaptopurine (6-MP), leflunomide, and malononitriloamides. Azathioprine is converted to a number of different metabolites that inhibit purine biosynthesis. Azathioprine and 6-MP are related chemically in that azathioprine is also converted into 6-MP inside the body. The 6-MP is incorporated into DNA of proliferating cells, thereby inhibiting DNA replication. Although used in high doses for reducing the risk organ transplant rejection and in treating leukemia, 6-MP at low doses may be used to treat inflammatory and autoimmune disorders, such as Crohn's disease and ulcerative colitis. Leflunomide is also an anti-metabolite affecting nucleic acid synthesis, but targets protein tyrosine kinases and dihydroorotate dehydrogenase, an enzyme critical to de novo pyrimidine biosynthesis. Malononitriloamides are inhibitors similar to the active metabolite of leflunomide, and therefore also act as pyrimidine biosynthesis inhibitors.

5.2.8 Anti-TNFα Agents

It is to be understood that anti-inflammatory agents other than those described above may be used in combination with the 2,4-pyrimidinediamine compounds. These include various agents directed against the cellular factors thought to be involved in promoting the inflammatory response. In some embodiments, the anti-inflammatory agent is an agent that blocks the action of TNF-α, the major cytokine implicated in inflammatory disorders. In some embodiments, the anti-TNF is an antibody that blocks the action of TNFα. An exemplary anti-TNF antibody is infliximab, available under the tradename Remicade®.

In other embodiments, the anti-TNFα agent is a receptor construct that binds TNFα and prevents its interaction with TNF receptors on present on cells. An exemplary anti-inflammatory agent based on TNFα receptor is entanercept, available under the tradename Enbrel®.

These and other anti-TNFα agents may be used alone with the 2,4-pyrimidinediamine compounds, or in combination with any of the other anti-inflammatory agents disclosed herein. For instance, etanercept in combination with an anti-metabolite anti-inflammatory agent, such as methotrexate, has been shown to be more effective in treating some autoimmune and inflammatory disorders (e.g., rheumatoid arthristis) than monotherapy with either agent.

### 5.3 Pharmaceutical Compositions and Administration

When used to treat or prevent such diseases, the compounds and anti-inflammatory agent may be administered singly, as mixtures of one or more 2,4-pyrimidinediamine compounds and one or more anti-inflammatory agents, or in mixture or combination with other agents useful for treating inflammatory diseases and/or the symptoms associated with inflammatory diseases. The 2,4-pyrimidinediamine compounds and anti-inflammatory agents may be administered per se, in the form of prodrugs or as pharmaceutical compositions.

Pharmaceutical compositions may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds and agents into preparations which can be used pharmaceutically. Guidance is provided in various reference works, such as Remington's Pharmaceutical Sciences, 1990, 18th Ed. (Gennard et al., eds.) Mack Publishing Company, and Gibson, M., Pharmaceutical Preformulation and Formulation: A Practical Guide from Candidate Drug Selection to Commercial Dosage Form, 2001, CRC Press.

The compounds and anti-inflammatory agents, or prodrugs thereof, may be formulated in the pharmaceutical compositions per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compounds and anti-inflammatory agents may be formulated as solutions, gels, ointments, creams, suspensions, etc. as are well-known in the art.

Systemic formulations include those designed for administration by injection, i.e., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the compounds and anti-inflammatory agents in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the active compound(s) maybe dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the 2,4-pyrimidinediamine compounds and anti-inflammatory agents, as is well known.

For buccal administration, the compositions may take the form of tablets or lozenges formulated in conventional manner.

For rectal and vaginal routes of administration, the compositions may be formulated as solutions (for retention enemas) suppositories or ointments containing conventional suppository bases such as cocoa butter or other glycerides.

For nasal administration or administration by inhalation or insufflation, the compounds and anti-inflammatory agents can be conveniently delivered in the form of an aerosol spray from pressurized packs or a nebulizer with the use of a suitable propellant, e.g.,) dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch. Description of metered doses inhalers are described in U.S. Pat. Nos. 6,532,955, 6,524,555, 6,251,368. Various formulations for inhalable forms of steroidal compounds are described in U.S. Pat. Nos. 4,835,142; 4,835,142, 4,906,476; and 5,192,528.

A specific example of an aqueous suspension formulation suitable for nasal administration using commercially-available nasal spray devices includes the following ingredients: 2,4-pyrimidinediamine compound (0.5-20 mg/ml); benzalkonium chloride (0.1-0.2 mg/mL); polysorbate 80 (TWEEN® 80 (0.5-5 mg/ml); carboxymethylcellulose sodium or microcrystalline cellulose (1-15 mg/ml); phenylethanol (1-4 mg/ml); and dextrose (20-50 mg/ml). The pH of the final suspension can be adjusted to range from about pH 5 to pH 7, with a pH of about pH 5.5 being typical.

For ocular administration, the compounds and anti-inflammatory agents may be formulated as a solution, emulsion, suspension, etc. suitable for administration to the eye. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851.

For prolonged delivery, the compound and anti-inflammatory agents can be formulated as a depot preparation for administration by implantation or intramuscular injection. The active ingredients may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compounds and agents for percutaneous absorption may be used. To this end, permeation enhancers may be used to facilitate transdermal penetration of the active compound(s). Suitable transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475.

Alternatively, other pharmaceutical delivery systems may be employed. Liposomes and emulsions are well-known examples of delivery vehicles that may be used to deliver active compound(s) or prodrug(s). In various embodiment, liposomes are formulated for site-specific release of entrapped compound into the inflamed region. Temperature sensitive liposomal formulations are described in, for example, U.S. Pat. No. 5,356,633. Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration.

The 2,4-pyrimidinediamine compounds and the anti-inflammatory agent may be administered in the form of a composition, or administered independently. When administered independently, the anti-inflammatory agents may be administered adjunctively, either concurrently or sequentially with the 2,4-pyrimidinediamine compounds. Administrations may be by the same route or by different routes. For instance, for treatment of allergic asthma, the 2,4-pyrimidinediamine compound may be administered by inhalation while the anti-inflammatory agent, such as a steroidal compound, is administered orally to provide more prolonged and systemic treatment. On the other hand, the 2,4-pyrimidinediamine compound and the steroidal anti-inflammatory agent may both be administered by inhalation to localize the treatment to the lungs. Determining the efficacious modes of administration will be within the skill of those in the art.

### 5.4 Effective Dosages

The 2,4-pyrimidinediamine compounds and anti-inflammatory agents are administered in combination to a subject afflicted with or at risk of developing an inflammatory disorder in an amount effective to treat the disorder. Generally, the compounds in combination will be used in an amount effective to achieve the intended result, for example in an amount effective to treat or prevent the particular disease being treated. The compound(s) may be administered therapeutically to achieve therapeutic benefit or prophylactically to achieve prophylactic benefit. By “therapeutic benefit” is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of the combination to a patient suffering from an allergy induced inflammation provides therapeutic benefit not only when the underlying allergic response is eradicated or ameliorated, but also when the patient reports a decrease in the severity or duration of the symptoms associated with the inflammatory condition. Therapeutic benefit also includes halting or slowing the progression of the disease, regardless of whether improvement is realized.

For prophylactic administration, the combination may be administered to a patient at risk of developing one of the previously described diseases or to prevent the recurrence of the symptoms or disorder. For example, prophylactic administration may be applied to avoid the onset of symptoms in a patient diagnosed with the underlying inflammatory disorder. 2,4-pyrimidinediamine compounds and anti-inflammatory agents may also be administered prophylactically to healthy individuals who are repeatedly exposed to agents known to one of the above-described maladies to prevent the onset of the disorder. For example, the combination may be administered to a healthy individual who is repeatedly exposed to an allergen or other insults known to induce inflammatory reaction.

The amount of combination administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Determining the dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmacological Basis of Therapeutics 10^{th} Ed. (Hardman et al., eds) Chapter 1, McGraw Hill (2001), and the references cited therein.

Initial dosages can also be estimated from in vivo data, such as animal models. Animal models useful for testing the efficacy of compounds to treat or prevent the various diseases described above are well-known in the art. Suitable animal models of hypersensitivity or allergic reactions, acute inflammation, and chronic inflammation are described in U.S. published patent applications 2004/0029902, 2005/0038243, 2005/0209224, 2005/0209230, 2005/0234049; Morgan and Marshall, 1999, In vivo models of inflammation, Birkhauser Verlag; Joe et al., 1999, Current Rheumatology Reports, 1:139-148; and Uchida M. and Mogami, O., 2005, J Pharmacol Sci. 97(2):285-8). Ordinarily skilled artisans can routinely adapt such information to determine dosages suitable for human administration.

Dosage amounts of the Syk inhibitory 2,4-pyrimidinediamine compounds will typically be in the range of from about 0.0001 or 0.001 or 0.01 mg/kg/day to about 100 mg/kg/day, but may be higher or lower, depending upon, among other factors, the activity of the compound, its bioavailability, the mode of administration and various factors discussed above. Dosage amount and interval may be adjusted individually to provide plasma or localized levels of the compound(s) which are sufficient to maintain therapeutic or prophylactic effect. Preferably, the compound(s) will provide therapeutic or prophylactic benefit without causing substantial toxicity. Toxicity of the compound(s) may be determined using standard pharmaceutical procedures. The dose ratio between toxic and therapeutic (or prophylactic) effect is the therapeutic index. Compounds(s) that exhibit high therapeutic indices are preferred.

The terms “effective amount” or “therapeutically effective amount” of the combination as provided herein is defined as an amount of the composition at least sufficient to provide the desired therapeutic effect. The exact amount required will vary from subject to subject, depending on age, general condition of the subject, the severity of the condition being treated, and the particular active agent administered, and the like.

In various embodiments, the anti-inflammatory agents are administered as normal approved dose (synonymously “standard dose”). As used herein, the term “normal approved dose” of an active agent as provided herein is defined as an amount of the agent that has been approved as safe and effective by the United States Food and Drug Administration for administration in humans in a particular dosage form. An approved dose is thus a dose found in a pharmaceutical product, an amount of active agent per unit dosage form. In general, reference to a ratio of approved doses means doses approved for the same patient population (e.g., adult to adult or pediatric to pediatric), and approved for the same dosage form (e.g., elixir, tablet, capsule, caplet, controlled release, etc.). Normal approved dosages for various anti-inflammatory agents, such as steroidal non-steroidal anti-inflammatory agents, may be found in various references, for example, Physicians Desk Reference, 2005, 59^{th} Ed., Thompson P D R, Montvale, N J, the disclosure of which is incorporated herein by reference.

In various embodiments, the 2,4-pyrimidinediamine compounds and anti-inflammatory agents, either as a composition or individually, may be administered once per week, several times per week (e.g., every other day), once per day or multiple times per day, depending upon, among other things, the mode of administration, the specific indication being treated, and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

As noted above, in certain cases a therapeutic amount of a 2,4-pyrimidinediamine compound may be administered to a patient in combination with a therapeutic amount of a second anti-inflammatory agent, e.g., a steroidal anti-inflammatory agent, a non-steroidal inflammatory agent, a lipoxygenase or 5-lipoxygenase activating protein (FLAP) antagonist, an anti-histamine, a β-agonist, an anti-metabolite anti-inflammatory agent, or an anti-TNFα agent, as described above. In such a combination therapy method, the 2,4-pyrimidinediamine compound may be co-administered with the second anti-inflammatory agent, either sequentially or simultaneously, or the 2,4-pyrimidinediamine compound and the second anti-inflammatory agent may be administered at different times. In certain cases, the 2,4-pyrimidinediamine compound and the second anti-inflammatory agent may be independently administered in the same, higher, or lower doses than if administered alone depending on such factors as, for example, the type of reagent used, the purpose for which the reagent and compound are being used, and clinical considerations. The 2,4-pyrimidinediamine compound and the second anti-inflammatory agent may be administered to a patient for period of time in the range of 4 weeks to 1 year or more, e.g., indefinitely.

In such a combination therapy, the 2,4-pyrimidinediamine compound may be administered to a subject at a daily dose of 50-500 mg/day. For example, the 2,4-pyrimidinediamine compound may be administered once a day, twice a day or three times a day. In particular embodiments, a dose of 50 mg to 150 mg (e.g., 100 mg) may be administered twice daily, or a dose of 100 mg to 300 mg (e.g., 150 mg) may be administered once daily.

The second anti-inflammatory agent may be administered at a dose that is consistent with the use of the second anti-inflammatory agent administered alone to a subject for the treatment of inflammation. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

In certain cases, treatment may be effected by administering an effective dosage of the second anti-inflammatory agent that that ranges from at least about 0.01 to 500 milligrams of the second anti-inflammatory agent per kilogram of patient per dose, e.g., from at least about 0.1 to 100 milligrams agent/kilogram of patient per single or multiple administration, depending upon the specific activity of the agent. In particular embodiments, the second anti-inflammatory agent can be administered once in a period of time in the range of one week to one month, e.g., once a week, once every other week or once per month, by injection, for example.

For example, if the second anti-inflammatory is methotrexate, the methotrexate may be administered in a combination therapy at a dose of 7.5-25 mg/week, e.g., 10-20 mg/week or about 15 mg/week. If the second anti-inflammatory is an anti-TNFα agent, e.g., an antibody such as etanercept, infliximab, or adalimumab or tocilizumab, the an anti-TNFα agent may be administered in a combination therapy at a dose of 10 mg/week to 200 mg/week. In particular embodiments, if the second anti-inflammatory agent is an anti-TNFα agent, the second anti-inflammatory agent may be administered at a dose of 10-100 mg (e.g., 20 mg, 40 mg or 60 mg) by subcutaneous injection every week, every other week, or every month.

In some embodiments, the patient to which the combination therapy is administered may be resistant to treatment with or intolerant to the second anti-inflammatory agent. In certain cases the patient to which combination therapy is administered has had active inflammation (for example, rheumatoid arthritis) for at least 1 year, and that has received a minimum of at least 6 months of therapy using the second anti-inflammatory agent (e.g., methotrexate at a dose of 7.5 to 25 mg/week), which, in certain cases, may further include a supplemental folic acid or folic acid therapy. In certain cases, the patient chosen for combination therapy may have at least 6 swollen joints and at least 6 tender joints (using the 28 joint count) and either an erythrocyte sedimentation rate of greater than 28 mm/hr, a C-reactive protein level greater than the upper limit of normal, or morning stiffness of at least 45 minutes duration, for example.

### 5.5 Inflammatory Diseases and Inflammatory Conditions

The compositions and method of the present disclosure may be used to treat a variety inflammatory diseases, or conditions in which an inflammatory response is associated with the disorder. Diagnosis and clinical indications of such diseases and conditions will be well known to the skilled artisan, and guidance is provided in various reference works, such as The Merck Manual of Diagnosis and Therapy, 1999, 17^{th} Ed., John Wiley & Sons; and International Classification of Disease and Related Health Problems (ICD 10), 2003, World Health Organization.

Acute and chronic inflammatory disorders that can be treated include, by way of example and not limitation, inflammatory conditions arising from atopy or anaphylactic hypersensitivity or allergic reactions, allergies (e.g., allergic conjunctivitis, allergic rhinitis, atopic asthma, atopic dermatitis and food allergies) and various inflammatory diseases characterized by tissue destruction and adverse tissue remodeling.

Exemplary inflammatory diseases or disorders that may be treated using the combination of a 2,4-pyrimidinediamine compound and an anti-inflammatory agent include, without limitation, asthma, lung inflammation, chronic granulomatous diseases such as tuberculosis, leprosy, sarcoidosis, and silicosis, nephritis, amyloidosis, rheumatoid arthritis, ankylosing spondylitis, chronic bronchitis, scleroderma, lupus, polymyositis, appendicitis, inflammatory bowel disease, Crohn's disease, ulcerative colitis, psoriasis, pelvic inflammatory disease, irritable bowel syndrome, orbital inflammatory disease, thrombotic disease, and inappropriate allergic responses to environmental stimuli such as poison ivy, pollen, insect stings and certain foods, including atopic dermatitis and contact dermatitis.

Inflammatory conditions associated with autoimmune diseases arising from any nonanaphylactic hypersensitivity reactions may be treated or prevented by use of the combination treatment. The compositions and methods may be used to treat or prevent those autoimmune diseases, and associated inflammatory conditions, frequently characterized as single organ or single cell-type autoimmune disorders including, but not limited to: Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis, Graves' disease, primary biliary cirrhosis, chronic aggressive hepatitis, ulcerative colitis and membranous glomerulopathy, as well as systemic autoimmune disorders, which include but are not limited to: systemic lupus erythematosis, rheumatoid arthritis, Sjogren's syndrome, Reiter's syndrome, polymyositis-dermatomyositis, systemic sclerosis, polyarteritis nodosa, multiple sclerosis and bullous pemphigoid. In other embodiments, the compositions and methods may be used to treat inflammation associated with malignant conditions, such as mastocytosis, fungoid mycosis (e.g., Sezary syndrome) and acute leukemia/lymphoma.

As a specific example of treatment, rheumatoid arthritis is thought to be an autoimmune disease that commonly affects the joints in a polyarticular manner (polyarthritis). The disease is characterized by chronically inflamed synovium that is densely crowded with lymphocytes. Chronic inflammatory condition arising from an autoimmune reaction can lead to led to erosion and destruction of the joint surface, which impairs the range of joint movement and leads to deformity. The 2,4-pyrimidinediamine compound in combination with the anti-inflammatory agents may be used to treat or ameliorate any one, several or all of these symptoms of rheumatoid arthritis.

An example involving an inhaled administration of the combination of 2,4-pyrimidinediamine compound and anti-inflammatory agent is the treatment of asthma. Asthma is a disease of the respiratory system in which the airways narrow, often in response to a stimuli such as exposure to an allergen, air irritant (e.g., ozone, nitrogen dioxide, sulfur dioxide), exercise, or emotional stress. In subjects with asthma, there appears to be abnormal levels of Th2 helper T cells and consequent activation of the humoral response. Upon exposure to a stimulus, such as an allergen, mast cells, basophils, and eosinophils in the airway epithelium are induced to release mediators, such as histamine, eicosanoids, and cytokines, which affect the mucosa of the airways, increasing mucosal edema, and mucus production, smooth muscle constriction, and recruitment other immune cells. The late phase of the asthmatic reaction is characterized by an influx of inflammatory and immune cells that secrete various cytokines and lipid mediators involved in hyper-responsiveness, mucus secretion, bronchoconstriction, and sustained inflammation. In various embodiments, the combination of the 2,4-pyrimidinediamine compound and anti-inflammatory agent (e.g., steroidal anti-inflammatory agent) may be prepared as an inhalable pharmaceutical composition administrable to the lungs upon manifestation of the asthma, or as a prophylactic measure to prevent occurrence of asthma.

It is to be understood that the person skilled in the art can apply the compositions and methods to treat a variety of other inflammatory conditions, and is not to be limited to the specific disorders described above.

The foregoing descriptions of specific embodiments of the present disclosure have been presented for purposes of illustration and description. They are not intended to be exhaustive or to limit the scope of the disclosure to the precise forms disclosed, and many modifications and variations are possible in light of the above teaching.

All patents, patent applications, publications, and references cited herein are expressly incorporated by reference to the same extent as if each individual publication or patent application was specifically and individually indicated to be incorporated by reference.

What is claimed is:

1. A composition for treating inflammatory disorders, comprising a Syk inhibitory 2,4-pyrimidinediamine compound and an anti-inflammatory agent.

2. The composition of claim 1 in which anti-inflammatory agent is a steroidal anti-inflammatory agent.

3. The composition of claim 2 in which the steroidal anti-inflammatory agent is a short acting steroid.

4. The composition of claim 3 in which the short acting steroid is selected from hydrocortisone, cortisone, and cortisol.

5. The composition of claim 2 in which the steroidal anti-inflammatory agent is an intermediate acting steroid.

6. The composition of claim 5 in which the intermediate acting steroid is selected from prednisone, prednisolone, triamcinolon, and methylprednisolone.

7. The composition of claim 2 in which the steroidal anti-inflammatory agent is a long acting steroid.

8. The composition of claim 7 in which the long acting steroid is selected from dexamethasone and betamethasone.

9. The composition of claim 2 in which the steroidal anti-inflammatory agent is a glucocorticosteroid.

10. The composition of claim 9 in which the glucocorticosteroid is selected from cortisol, cortisone, prednisone, prednisolone, triamcinolone, methylprednisolone, dexamethasone, and betamethasone.

11. The composition of claim 9 in which the glucocorticosteroid has minimal mineralocorticosteroid activity.

12. The composition of claim 11 in which the glucocorticosteroid is selected from methylpredisolone, triamcinolone, betamethasone, and dexamethasone.

13. The composition of claim 2 in which the steroidal anti-inflammatory agent is a mineralocorticosteroid.

14. The composition of claim 13 in which the mineralocorticosteroid is fludrocortisone.

15. The composition of claim 2 in which the steroidal anti-inflammatory agent is an inhalable steroid composition.

16. The composition of claim 15 in which the inhalable steroidal composition is selected from beclomethasone, flunisolide, triamcinolone, fluticasone, dexamethasone, and budesonide.

17. The composition of claim 1 in which the anti-inflammatory agent is a non-steroidal anti-inflammatory agent.

18. The composition of claim 17 in which the non-steroidal anti-inflammatory agent is a Cox inhibitor.

19. The composition of claim 18 in which the Cox inhibitor is a non-selective Cox inhibitor.

20. The composition of claim 19 in which the non-selective Cox inhibitor is a salicylic acid, indole acetic acid, indene acetic acid, heteroacryl acetic acid, arylpropionic acid, anthranilic acid, enolic acid, alkanone, or derivative thereof.
